(19) United States
(12) Patent Application Publication
(10) Pub. No.: US 2010/0203553 A1
(43) Pub. Date: Aug. 12, 2010

(54) Histochemical and biomarker for liver fibrosis

(76) Inventors: Suad M. Abdeen, Safat (KW); Samuel O. Olusi, Safat (KW)

Correspondence Address: LITMAN LAW OFFICES, LTD., POST OFFICE BOX 41200, SOUTH STATION, ARLINGTON, VA, 22204, US

(21) Appl. No.: 12/379,045

(22) Filed: Feb. 11, 2009

(51) Int. Cl. G01N 33/573; G01N 1/30

(52) U.S. Cl. 435/7.4; 435/40.52

(57) ABSTRACT

The histochemical and biomarker for liver fibrosis is a technique for determining the existence and extent of liver fibrosis by evaluating the extent of peptidylarginine deiminase (PAD) activity by immunological methods. The method is illustrated using liver biopsy. A small section of tissue from a liver biopsy is incubated overnight with a monoclonal antibody specific to PAD, stained, and examined microscopically. The number of hepatocytes that positively express PAD activity per one hundred hepatocytes is counted and expressed as a percentage. The percentage of PAD activity is then correlated with a METAVIR fibrosis score according to results from a statistically significant reference population. The degree of liver fibrosis and an appropriate treatment regimen may then be determined.

### BACKGROUND OF THE INVENTION

1. Field of the Invention

The present invention relates to methods for diagnosing liver fibrosis, and particularly to a histochemical and biomarker for liver fibrosis that provides a way of diagnosing and monitoring the stage of liver fibrosis in order to prescribe and maintain treatment regimens for conditions affecting the liver.

2. Description of the Related Art

Liver fibrosis, a characteristic of most types of chronic liver diseases, results from chronic damage to the liver together with the accumulation of extracellular matrix (ECM) proteins. The main causes of liver fibrosis in most countries of the world are chronic hepatitis C virus (HCV) infection, alcohol abuse, and non-alcoholic steatohepatitis (NASH). The accumulation of ECM proteins distorts the hepatic architecture by forming a fibrous scar, and the subsequent development of nodules of regenerating hepatocytes results in cirrhosis, which may eventually lead to organ failure and death. Often, determining the extent of the progression of the condition and a suitable treatment regimen for retarding or reversing the disease process requires a determination of the extent of liver fibrosis.

The activation of hepatic stellate cells (HSCs) is a critical event in hepatic fibrosis. In the normal liver, most HSCs, also called Ito cells, are in a resting state. In response to liver injury, these cells are activated and undergo structural and functional changes, which include the expression of cell surface receptors, increased cell proliferation, and the synthesis of extracellular matrix (ECM) proteins. Moreover, activated HSCs can contribute to hepatic inflammation by their ability to secrete and respond to a wide range of growth factors. The exact mechanism responsible for the activation of these cells is not fully known.

One system for grading the extent of liver fibrosis is the METAVIR system. The system provides for grading the extent of fibrosis from F0 through F4 as follows: F0=no scarring; F1=minimal scarring; F2=scarring has occurred and extends outside the areas of the liver that contain blood vessels; F3=bridging fibrosis is spreading and connecting to other areas that contain fibrosis; and F4=cirrhosis or advanced scarring of the liver. In addition to the presence of fibrotic tissue, the extent of fibrosis is sometimes also evaluated according to the degree of inflammation present, referred to as the grade or activity level. Various systems for evaluating the activity level are used, including the Knodell score, METAVIR, etc. In the METAVIR system, activity ranges from A0 to A3, as follows: A0—no activity; A1—minimal activity; A2—moderate activity; and A3—severe activity.

The most frequently used test to evaluate the level of fibrosis is the liver biopsy. In liver biopsy, a section of tissue is excised, stained, examined by microscopy, and graded according to the above systems to evaluate the extent of fibrosis. While pathological evaluation of tissue samples is standard medical treatment, nevertheless, there are problems associated with liver biopsies. The biopsy is an invasive procedure, resulting in cost and inconvenience to the patient. The tissue sample is small (about 1:50,000 of the liver), and since some forms of fibrosis are patchy, the sample may not be representative. Finally, the grading systems mentioned above tend to be somewhat subjective, depending upon the experience and skill of the pathologist evaluating the appearance of the stained tissue to maintain consistency in the evaluation. The process is also cumbersome and time consuming.

Peptidylarginine deiminases (PAD or PADI) are a family of enzymes involved in the posttranslational deimination of protein-bound arginine to citrulline. Deimination of proteins decreases the net positive charge, alters intra- and intermolecular ionic interactions, and probably the folding of target proteins. Five paralogous genes encode the five human peptidylarginine deiminase (PAD) isoforms, PADI 1,2,3,4, and 6.

The genes PADI are clustered on chromosome Ip 35-36, a region conserved with synteny on the mouse chromosome 4E 1. The presence of citrulline-modified target epitopes for autoantibodies is a well-known phenomenon in rheumatoid arthritis. PADS were recently implicated in the generation of anticyclic citrullinated peptide antibodies (antiCCP) detectable in early stages of rheumatoid arthritis (RA). The process resulting in anti-CCP formation is thought to play a pivotal role in the early stages of RA evolution, since it is detectable several years before the onset of symptoms. The association of PAD activity, especially of PAD 4, with a number of other inflammatory diseases, such as multiple sclerosis, systemic lupus erythematosus, and primary Sjogren syndrome suggests the involvement of this enzyme in cellular stress mechanisms.

The presence of peptidylarginine deiminase in tissue samples may be determined by immunohistochemical staining techniques. Moreover, the level of activity of PAD may be determined by various systems for grading the intensity of the immunohistochemical staining. In view of the subjectivity of conventional liver biopsy techniques for evaluating the existence and extent of liver fibrosis, and in view of the cumbersome and time-consuming nature of the process, it is desirable to provide a more objective, easier process to determine the existence and extent of liver fibrosis. Thus, a biomarker for liver fibrosis solving the aforementioned problems is desired.

### SUMMARY OF THE INVENTION

The histochemical marker for liver fibrosis is a technique for determining the existence and extent of liver fibrosis by evaluating the extent of peptidylarginine deiminase (PAD) activity by immunohistochemical methods. The method is illustrated using liver biopsy. A small section of tissue from a liver biopsy is incubated overnight with a monoclonal antibody specific to PAD, stained, and examined microscopically. The number of hepatocytes that positively express PAD activity per one hundred hepatocytes is counted and expressed as a percentage. The percentage of PAD activity is then correlated with a METAVIR fibrosis score according to results from a statistically significant reference population. The degree of liver fibrosis and an appropriate treatment regimen may then be determined.

As noted above, the exact mechanism for activation of hepatic stellate cells (HSC) is not fully known. The present inventors hypothesize that the enzyme peptidylarginine deiminase may activate the HSCs. The association of PAD activity with inflammatory diseases, such as rheumatoid arthritis (RA), multiple sclerosis (MS), systematic lupus erythematosus (SLE), and primary Sjogren syndrome, suggests that the PAD enzyme is involved in cellular stress mechanisms. Since liver fibrosis involves cellular stress and deposition of extracellular matrix (ECM), just as in RA and MS, the inventors hypothesize that PAD may be involved in its pathogenesis. Furthermore, since immunohistochemical testing of liver biopsy samples demonstrates a correlation between the level of PAD activity and the degree of liver fibrosis, it should be possible to diagnose the presence and degree of liver fibrosis based upon immunohistochemical demonstration of PAD in liver biopsies and the biochemical or ELISA estimation of PAD in serum and liver. It should be noted that the present invention relates to (i) the immunohistochemical staining of PAD in liver biopsies for staging and grading liver fibrosis; and (ii) the immunoassay of PAD in serum as a non-invasive biomarker for staging and grading liver biopsies.

These and other features of the present invention will become readily apparent upon further review of the following specification and drawings.

### BRIEF DESCRIPTION OF THE DRAWINGS

FIG. 1 is a chart showing the percentage of liver cells per one hundred hepatic cells evidencing PAD expression versus the METAVIR fibrosis score in one hundred patients by immunohistochemical testing of liver biopsy samples.

FIG. 2 is a chart showing the mean CD38 positive hepatic stellate cells (HSC) per 10 hpf vs. the percentage of hepatic cells expressing PAD activity in one hundred patients by immunohistochemical testing of liver biopsy samples.

Similar reference characters denote corresponding features consistently throughout the attached drawings.

### DETAILED DESCRIPTION OF THE PREFERRED EMBODIMENTS

The biomarker for liver fibrosis is a technique for determining the existence and extent of liver fibrosis by evaluating the extent of peptidylarginine deiminase (PAD) activity by immunological methods. The method is illustrated using liver biopsy. A small section of tissue from a liver biopsy is incubated overnight with a monoclonal antibody specific to PAD, stained, and examined microscopically. The number of hepatocytes that positively express PAD activity per one hundred hepatocytes is counted and expressed as a percentage. The percentage of PAD activity is then correlated with a METAVIR fibrosis score according to results from a statistically significant reference population. The degree of liver fibrosis and an appropriate treatment regimen may then be determined.

As noted above, the exact mechanism for activation of hepatic stellate cells (HSC) is not fully known. The present inventors hypothesize that the enzyme peptidylarginine deiminase may activate the HSCs. The association of PAD activity with inflammatory diseases, such as rheumatoid arthritis (RA), multiple sclerosis (MS), systematic lupus erythematosus (SLE), and primary Sjogren syndrome, suggests that the PAD enzyme is involved in cellular stress mechanisms. Since liver fibrosis involves cellular stress and deposition of extracellular matrix (ECM), just as in RA and MS, the inventors hypothesize that PAD may be involved in its pathogenesis. Furthermore, since immunohistochemical testing of liver biopsy samples demonstrates a correlation between the level of PAD activity and the degree of liver fibrosis, it should be possible to diagnose the presence and degree of liver fibrosis based upon the biochemical and immunochemical estimation of PAD in serum.

The following example demonstrates how peptidylarginine deiminase (PAD) may be used as a biomarker for liver fibrosis using immunohistochemical testing of liver biopsy samples.

### EXAMPLE

Patients. One hundred patients with chronic liver diseases of varying durations who reported for liver biopsy at the hepatology clinics of Al-Amiri and Mubarak Al-Kabeer University Teaching Hospitals, Kuwait between September 2006 and November 2007 were recruited for this study. Theses patients had reported for liver biopsy because of past history of jaundice and persistently elevated serum transaminases. All of the patients gave informed consent for use of data and serum for research purposes, and this study was approved by the local research ethical committee.

Histological Analysis of Liver Biopsies. Liver biopsies were formalin fixed, paraffin-embedded and stained with H&E stain, as well as other special stains used for liver tissue diagnosis, such as Masson's trichrome stain for collagen, reticulin stain, PAS stain with and without diastase digestion, Perl's iron stain and orcein stain. Two histopathologists, completely unaware of patient characteristics, examined the biopsies under the microscope to assess the degree of fibrosis (stage) and extent of inflammatory activity (grade) according to the METAVIR scoring system (21). Fibrosis was staged on a scale of 0-4: F0=no fibrosis; FI=portal fibrosis without septa; F2=few septa; F3=numerous septa without fibrosis; F4=complete fibrosis (cirrhosis). The grading of activity assessed by the METAVIR system (based on the intensity of necroinflammatory activity, interface hepatitis and lobulitis) was scored as follows: A0=no histological inflammatory activity; A1=mild inflammatory activity; A2=moderate inflammatory activity; A3 =severe inflammatory activity (21). To assess liver biopsy quality, Regev quality criteria (22) were used. A biopsy between 10 and 15 mm in length, with less than five portal tracts or fragmented is considered as “fair quality biopsy”. ‘A poor quality’ biopsy is under 10 mm in length. All poor quality biopsies were excluded from the study.

Immunohistochemical Staining for PAD: The kit for the immunohistochemical demonstration of peptidylarginine deiminase was obtained from Novos Biologicals, USA. The instructions of the manufacturers of the kit were followed. Formalin fixed and paraffin-embedded 4/lm sections of liver biopsies were used. Tissue sections (liver, heart muscle, brain, skin, brain choroidal plexus) were deparaffinized in xylene and hydrated in graded series of ethanol solutions. After a rinse in phosphate buffered saline (PBS), endogenous peroxidase activity was quenched by incubating sections for 10 minutes in 0.3% H₂O₂ in absolute methanol. Following a 10-minute rehydration in PBS, the sections were preheated using pressure cooker for 20 minutes in 10 mM citrate buffer (PH 6.0) for antigen retrieval. The sections were then incubated with blocking serum (4% normal horse serum) for 30 minutes at room temperature. The sections were then incubated in 1/200 dilution of monoclonal antibody to PAD (Novus Biologicals, USA) for overnight and treated with a biotinylated goat anti-mouse antibody for 30 minutes. The sections were then washed thrice in PBS and incubated with an avidin-biotinylated horseradish peroxidase macromolecular complex for 30 minutes. The stained sections were visualized with 3,3′-diaminobenzidine tetrachloride, and the slides were counterstained with Mayer's hematoxylin. Finally, the sections were dehydrated in a series of ethanol solutions and cleared with xylene. The intensity of PAD staining in the biopsies was graded according to the method of Zlobec et al, i.e., per every 100 hepatocytes, the hepatocytes that positively expressed the PAD enzyme were counted and the percentages were calculated. Very weak expressions were excluded.

Statistical Analysis: Data entry and statistical analysis were performed using SPSS version 15.0. We evaluated the association between PAD activity and METAVIR fibrosis and activity scores. We used error bar plots with 95% confidence intervals to display these associations. Mann-Whitney U test was used to compare PAD activity with various grades of activity and degrees of fibrosis to find out if PAD activity could significantly predict the stage or grade of liver fibrosis.

Patient characteristics. The mean age of the 100 patients with chronic hepatitis was 47.39±8.40 years with a male preponderance (see Table I). All of the patients had significantly elevated concentrations of serum alanine aminotransaminase (ALT) and aspartate aminotransaminase (AST) with the concentrations of these enzymes rising with the grade of activity and degree of liver fibrosis. Forty-six (46%) patients had hepatitis C virus (HCV) infection, 20 (20%) hepatitis B virus (HBV) infection, 8 (8%) non-alcoholic steato-hepatitis (NASH), 8 (8%) autoimmune hepatitis, 2 (2%) fibrosing cholestatic hepatitis, 8 (8%) other infections and 8 were normal. Seventy-two (72%) of the patients had significant fibrosis (FI, F3 or F4), suggesting that most of the patients presented late for liver biopsy. Furthermore, significant fibrosis was present more often in patients with hepatitis C virus (HCV) than those with hepatitis B virus (HBV) (38% vs. 14%). This difference was found to be statistically significant (p<0.05).

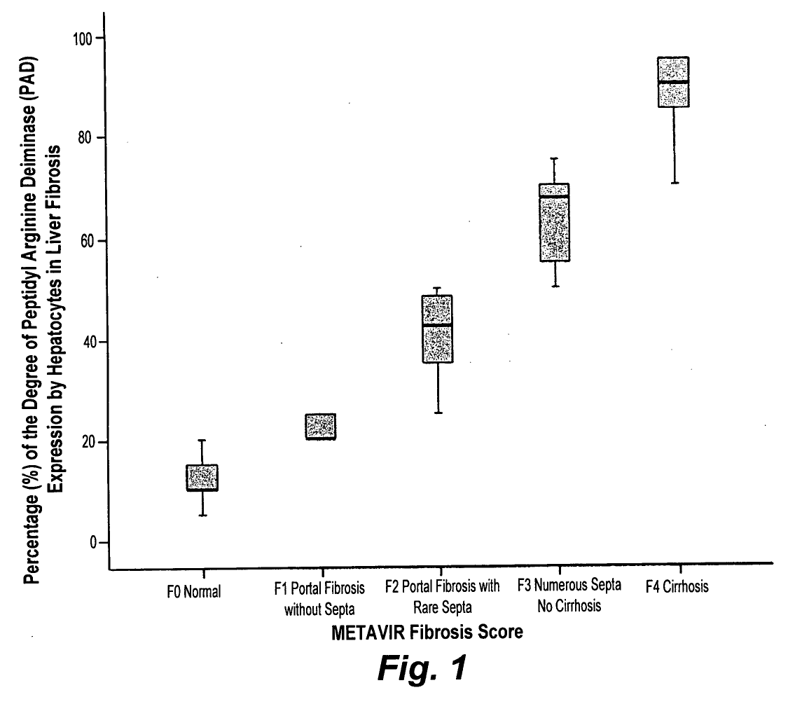

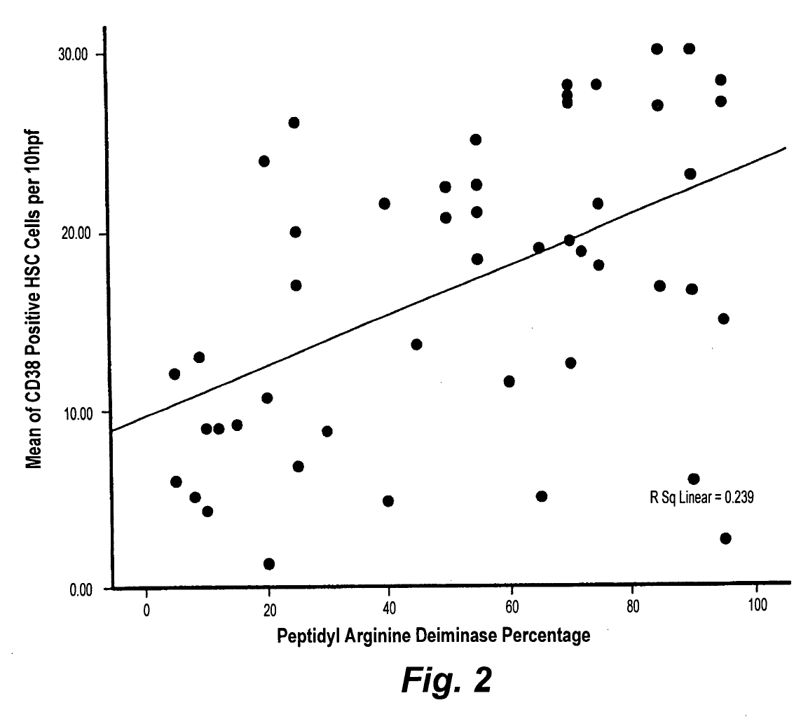

Immunohistochemical demonstration of PAD in liver biopsies: Microscopic examination of the stained liver biopsy samples showed that PAD could be demonstrated within the hepatocytes. PAD activity was confirmed in other tissues known to contain the enzyme, such as the skin, brain, heart muscle and choroidal plexus, also by staining tissue samples immunohistochemically with subsequent microscopic examination. Similar examination showed that PAD is absent in the hepatocytes in patients without liver fibrosis (F0 METAVIR). As shown in FIGS. 1 and 2, PAD activity in the hepatocytes increases with the degree of hepatic fibrosis in patients with chronic liver disease. Thus, PAD activity in patients with F4 fibrosis was significantly greater than those with FI, F2, or F3 fibrosis.

TABLE I
Demographic and Biochemical Characteristics of Patients

| | | | Mean | | | | | | | |
|---|---|---|---|---|---|---|---|---|---|---|
| METAVIR | | | Age | ALT | AST | | | | | |
| Fibrosis | n | M/F | (yr) | (IU/L) | (IU/L) | HCV | HBV | NASH | AIH | Others |
| F0 | 18 | 15/3 | 41.89 ± 4.16 | 69.2 ± 57.7 | 57.75 ± 12.52 | 4 | 2 | 0 | 0 | 12 |
| F1 | 10 | 8/2 | 46.8 ± 1.65 | 79.85 ± 15.3 | 70.43 ± 16.21 | 4 | 4 | 0 | 2 | 0 |
| F2 | 14 | 10/4 | 44 ± 1.31 | 84.25 ± 25.23 | 75.71 ± 13.07 | 10 | 2 | 0 | 0 | 2 |
| F3 | 34 | 24/10 | 45.47 ± 2.05 | 180.6 ± 25.8 | 136.67 ± 84.06 | 16 | 6 | 4 | 6 | 2 |
| F4 | 24 | 17/7 | 42.8 ± 1.54 | 191.63 ± 78.39 | 145.18 ± 59.23 | 12 | 6 | 4 | 0 | 2 |
| Total | 100 | 74/26 | 44.14 ± 11.09 | 93.88 ± 16.46 | 114.62 ± 22.69 | 46 | 20 | 8 | 8 | 18 |

n = number of patients
Mean age ± S.D.
ALT = Alanine aminotransaminase
AST = Aspartate aminotransaminase
HCV = Hepatitis C Virus
HBV = Hepatitis B Virus
NASH = Non-Alcoholic Steato-Hepatitis
AIH = Autoimmune hepatitis
Others (Normal (8), Fibrosing Cholestatic Hepatitis (2), other infections (8))

In particular, PAD expression in between about 0% and 20%, and more particularly between about 10% and 15%, of hepatocytes correlates with a METAVIR fibrosis score of F0 (normal); PAD expression in between about 20% and 25% of hepatocytes correlates with a METAVIR fibrosis score of F1 (portal fibrosis without septa); PAD expression in between about 25% and 50%, and more particularly between about 35% and 45%, of hepatocytes correlates with a METAVIR fibrosis score of F2 (portal fibrosis with rare septa); PAD expression in between about 50% and 80%, and more. particularly between about 55% and 70%, of hepatocytes correlates with a METAVIR fibrosis score of F3 (numerous septa but no cirrhosis); and PAD expression in between about 75% and 100%, and more particularly between about 85% and 95%, of hepatocytes correlates with a METAVIR fibrosis score of F4 (cirrhosis).

In addition, PAD activity was also able to significantly discriminate between F0 and F1, between F1 and F2, between F2 and F3, and between F3 and F4 liver fibrosis (see Table II for the statistical significance).

TABLE II
Significance of correlation between PAD expression and METAVIR
scores

| Outcome | | | |
|---|---|---|---|
| Variable | Contrasts | P-Value | r-value |
| Degree of | None (A0) versus Mild | <0.003 | 0.693 |
| inflammation | (A1) | | |
| | None (A0) versus | <0.001 | |
| | Moderate (A2) | | |
| | None (A0) versus | <0.001 | |
| | Severe (A3) | | |
| | Mild (A1) versus | <0.001 | |
| | Moderate (A2) | | |
| | Moderate (A2) versus | <0.258 | |
| | Severe (A3) | | |
| Fibrosis | None (F0) versus PF- | <0.001 | 0.944 |
| | no septa (F1) | | |
| | None (F0) versus PF- | <0.001 | |
| | rare septa (F2) | | |
| | None (F0) versus PF- | <0.001 | |
| | many septa (F3) | | |
| | None (F0) versus PF- | <0.001 | |
| | cirrhosis (F4) | | |
| | No septa (F1) versus PF | <0.058 | |
| | rare septa (F2) | | |
| | Rare septa (F2) versus | <0.002 | |
| | PF-many septa (F3) | | |
| | Many septa (F3) versus | <0.001 | |
| | PF-cirrhosis (F4) | | |

Associations between PAD activity and METAVIR grade: The PAD activity level was significantly lower in liver biopsies of patients with no hepatic inflammation (A0) than in those with mild (A1), moderate (A2) or severe activity (A3). Additionally PAD activity was able to statistically distinguish patients with no chronic hepatic inflammation from those with either mild or moderate or severe inflammation.

Our results showed that PAD could be demonstrated immunohistochemically in liver biopsies from patients with chronic liver disease and that its degree of activity could differentiate patients with no inflammation or fibrosis from those with different grades of inflammation or stages of fibrosis. Liver fibrosis is associated with major alterations in both the quantity and composition of ECM. In advanced stages, the liver contains approximately six times more ECM than normal, including collagens, fibronectin, undulin, elastin, laminin, hyaluronan and proteoglycans. Accumulation of ECM results from both increased synthesis and decreased degradation.

HSCs are the main ECM producing cells in the injured liver. Following chronic injury HSCs activate or transdifferentiate into myofibroblast-like cells, acquiring contractile, proinflammatory and fibrogenic properties. Activated HSCs migrate and accumulate at the sites of tissue repair, secreting large amounts of ECM and regulating ECM degradation. Although PDGF, mainly produced by Kupfer cells, is said to be the predominant mitogen for activated HSCs, we now provide evidence that the enzyme PAD may also be important in this activation, since we showed that the enzyme was associated both with the degree of hepatic inflammation and the stage of fibrosis.

The exact mechanism by which PAD activates HSCs is not at present known. But it is known that PAD4 can translocate into the nucleus in which it deiminates histones. This histone deimination within the nucleus may lead to cell activation. Since the HSCs contain fibronectin, which is rich in histones, it is probable that this process may contribute to their activation. Further work is required to elucidate the mechanism of activation of HSCs by PAD.

We conclude that immunohistochemical demonstration of PAD activity in liver biopsies can be used to grade hepatic inflammation and fibrosis in patients with chronic hepatitis. It should also be possible to grade hepatic inflammation and fibrosis in patients with chronic hepatitis on the basis of biochemical and immunochemical estimates of PAD activity in serum.

It is to be understood that the present invention is not limited to the embodiments described above, but encompasses any and all embodiments within the scope of the following claims.

We claim:

1. A method for diagnosing liver fibrosis and its extent in patients, comprising the steps of:
immunochemically testing peptidylarginine deiminase levels in the patient; and
comparing the patient's peptidylarginine deiminase levels with peptidylarginine deiminase levels in a reference population having known liver fibrosis scores ranging from normal through cirrhosis of the liver.

2. The method for diagnosing liver fibrosis according to claim 1, wherein said step of immunochemically testing peptidylarginine deiminase levels comprises immunochemically testing peptidylarginine deiminase levels in serum.

3. The method for diagnosing liver fibrosis according to claim 1, wherein said step of immunochemically testing peptidylarginine deiminase levels comprises immunohistochemically testing peptidylarginine deiminase levels in a sample of tissue obtained by liver biopsy.

4. The method for diagnosing liver fibrosis according to claim 3, wherein said step of immunohistochemically testing peptidylarginine deiminase levels in a sample of tissue obtained by liver biopsy further comprises the steps of:
incubating a small section of tissue from a liver biopsy overnight with a monoclonal antibody specific to peptidylarginine deiminase;
staining the incubated tissue section; and
examining the stained tissue section microscopically.

5. The method for diagnosing liver fibrosis according to claim 4, wherein said step of immunohistochemically testing peptidylarginine deiminase levels in a sample of tissue obtained by liver biopsy further comprises the steps of counting the number of hepatocytes expressing peptidylarginine deiminase and expressing the number counted as a percentage.

6. The method for diagnosing liver fibrosis according to claim 5, wherein said the known liver fibrosis scores comprise METAVIR scores.

7. The method for diagnosing liver fibrosis according to claim 6, further comprising the step of:
diagnosing the patient with a METAVIR score of F0 (normal) when the patient's percentage of hepatocytes expressing peptidylarginine deiminase is between about 0% and 20%;
diagnosing the patient with a METAVIR score of F1 (portal fibrosis without septa) when the patient's percentage of hepatocytes expressing peptidylarginine deiminase is between about 20% and 25%;
diagnosing the patient with a METAVIR score of F2 (portal fibrosis with rare septa) when the patient's percentage of hepatocytes expressing peptidylarginine deiminase is between about 25% and 50%;
diagnosing the patient with a METAVIR score of F3 (numerous septa but no cirrhosis) when the patient's percentage of hepatocytes expressing peptidylarginine deiminase is between about 50% and 80%; and
diagnosing the patient with a METAVIR score of F4 (cirrhosis) when the patient's percentage of hepatocytes expressing peptidylarginine deiminase is between about 80% and 100%.

8. The method for diagnosing liver fibrosis according to claim 7, wherein said step of diagnosing the patient with a METAVIR score of F2 further comprises diagnosing the patient with a METAVIR score of F2 when the patient's percentage of hepatocytes expressing peptidylarginine deiminase is between about 35% and 45%.

9. The method for diagnosing liver fibrosis according to claim 7, wherein said step of diagnosing the patient with a METAVIR score of F3 further comprises diagnosing the patient with a METAVIR score of F3 when the patient's percentage of hepatocytes expressing peptidylarginine deiminase is between about 55% and 70%.

10. The method for diagnosing liver fibrosis according to claim 7, wherein said step of diagnosing the patient with a METAVIR score of F4 further comprises diagnosing the patient with a METAVIR score of F4 when the patient's percentage of hepatocytes expressing peptidylarginine deiminase is between about 85% and 95%.

11. A method for diagnosing liver fibrosis and its extent in patients, comprising the steps of:
immunohistochemically testing peptidylarginine deiminase levels in the patient in a sample of tissue obtained by liver biopsy; and
comparing the patient's peptidylarginine deiminase levels with peptidylarginine deiminase levels in a reference population having known liver fibrosis scores ranging from normal through cirrhosis of the liver.

12. The method for diagnosing liver fibrosis according to claim 11, wherein said step of immunohistochemically testing peptidylarginine deiminase levels in a sample of tissue obtained by liver biopsy further comprises the steps of:
incubating a small section of tissue from a liver biopsy overnight with a monoclonal antibody specific to peptidylarginine deiminase;
staining the incubated tissue section; and
examining the stained tissue section microscopically.

13. The method for diagnosing liver fibrosis according to claim 11, wherein said step of immunohistochemically testing peptidylarginine deiminase levels in a sample of tissue obtained by liver biopsy further comprises the steps of counting the number of hepatocytes expressing peptidylarginine deiminase and expressing the number counted as a percentage.

14. The method for diagnosing liver fibrosis according to claim 13, wherein said the known liver fibrosis scores comprise METAVIR scores.

15. The method for diagnosing liver fibrosis according to claim 14, further comprising the step of:
diagnosing the patient with a METAVIR score of F0 (normal) when the patient's percentage of hepatocytes expressing peptidylarginine deiminase is between about 0% and 20%;
diagnosing the patient with a METAVIR score of F1 (portal fibrosis without septa) when the patient's percentage of hepatocytes expressing peptidylarginine deiminase is between about 20% and 25%;
diagnosing the patient with a METAVIR score of F2 (portal fibrosis with rare septa) when the patient's percentage of hepatocytes expressing peptidylarginine deiminase is between about 25% and 50%;
diagnosing the patient with a METAVIR score of F3 (numerous septa but no cirrhosis) when the patient's percentage of hepatocytes expressing peptidylarginine deiminase is between about 50% and 80%; and
diagnosing the patient with a METAVIR score of F4 (cirrhosis) when the patient's percentage of hepatocytes expressing peptidylarginine deiminase is between about 80% and 100%.

16. The method for diagnosing liver fibrosis according to claim 15, wherein said step of diagnosing the patient with a METAVIR score of F2 further comprises diagnosing the patient with a METAVIR score of F2 when the patient's percentage of hepatocytes expressing peptidylarginine deiminase is between about 35% and 45%.

17. The method for diagnosing liver fibrosis according to claim 15, wherein said step of diagnosing the patient with a METAVIR score of F3 further comprises diagnosing the patient with a METAVIR score of F3 when the patient's percentage of hepatocytes expressing peptidylarginine deiminase is between about 55% and 70%.

18. The method for diagnosing liver fibrosis according to claim 15, wherein said step of diagnosing the patient with a METAVIR score of F4 further comprises diagnosing the patient with a METAVIR score of F4 when the patient's percentage of hepatocytes expressing peptidylarginine deiminase is between about 85% and 95%.
